(19) United States
(12) Patent Application Publication
(10) Pub. No.: US 2024/0016474 A1
(43) Pub. Date: Jan. 18, 2024

(54) IMAGE PROCESSING DEVICE, IMAGE PROCESSING SYSTEM, IMAGE DISPLAY METHOD, AND IMAGE PROCESSING PROGRAM

(71) Applicant: TERUMO KABUSHIKI KAISHA, Tokyo (JP)

(72) Inventors: Yasukazu SAKAMOTO, Hiratsuka-shi (JP); Katsuhiko SHIMIZU, Fujinomiya-shi (JP); Hiroyuki ISHIHARA, Tokyo (JP); Shunsuke YOSHIZAWA, Ebina-shi (JP)

(73) Assignee: TERUMO KABUSHIKI KAISHA, Tokyo (JP)

(21) Appl. No.: 18/473,370

(22) Filed: Sep. 25, 2023

Related U.S. Application Data
(63) Continuation of application No. PCT/JP2022/009242, filed on Mar. 3, 2022.

(30) Foreign Application Priority Data
Mar. 26, 2021 (JP) 2021-054095

(51) Int. Cl. A61B 8/00

(52) U.S. Cl. CPC A61B 8/4254; A61B 8/463

(57) ABSTRACT

An image processing device is an image processing device that displays an image representing a biological tissue on a display and displays an element representing a position of a sensor on a screen same as the image on the display, based on tomographic data acquired by the sensor moving in a lumen of the biological tissue, the image processing device including a control unit that, when a user operation of specifying a movement destination of the sensor is performed on the screen, controls movement of the sensor according to the user operation, and changes a relative position of the element to cause a position of the sensor after movement to be represented.

### CROSS-REFERENCES TO RELATED APPLICATIONS

This application is a continuation of International Application No. PCT/JP2022/009242 filed on Mar. 3, 2022, which claims priority to Japanese Application No. 2021-054095 filed on Mar. 26, 2021, the entire content of both of which is incorporated herein by reference.

### TECHNOLOGICAL FIELD

The present disclosure generally relates to an image processing device, an image processing system, an image display method, and an image processing program.

### BACKGROUND DISCUSSION

U.S. Patent Application Publication No. 2010/0215238 A, U.S. Pat. Nos. 6,385,332, and 6,251,072 describe a technique for generating a three-dimensional image of a cardiac cavity or a blood vessel using an ultrasound (US) imaging system.

Treatment using intravascular ultrasound (IVUS) is widely executed for a cardiac cavity, a cardiac blood vessel, a lower limb artery region, and the like. IVUS is a device or a method for providing a two-dimensional image of a plane perpendicular to a longitudinal axis of a catheter.

IVUS is often used for procedures using a catheter separate from an IVUS catheter, such as ablation. Basically, a procedure is often performed by a team including staff members such as doctors and clinical engineers, but a procedure may be performed in a state where the number of staff members is relatively small. In such a case, it can be difficult for a doctor performing a procedure while operating a catheter or a clinical engineer operating an IVUS system while observing a display to operate an IVUS pullback unit.

### SUMMARY

The present disclosure enables operation of a sensor on a screen.

An image processing device as one aspect of the present disclosure is an image processing device that displays an image representing a biological tissue on a display and displays an element representing a position of a sensor on a screen same as the image on the display, based on tomographic data acquired by the sensor moving in a lumen of the biological tissue, the image processing device including a control unit that, when a user operation of specifying a movement destination of the sensor is performed on the screen, controls movement of the sensor according to the user operation, and changes a relative position of the element to cause a position of the sensor after movement to be represented.

In an embodiment, the control unit receives the user operation in a case in which a pre-operation of clicking or tapping the element while pressing a predetermined key is performed before the user operation.

In an embodiment, the control unit receives, as the user operation, an operation of dragging the element and dropping the element at a position corresponding to a movement destination of the sensor.

In an embodiment, the control unit controls movement of the sensor after the element is dropped.

In an embodiment, the control unit adjusts a movement speed of the sensor, and causes the sensor to reach a movement destination in a certain period of time regardless of a distance from a position of the element before dragging to a position of the element after dropping.

In an embodiment, the control unit controls movement of the sensor while the element is being dragged.

In an embodiment, the control unit limits a speed at which the element is dragged according to an upper limit of a movement speed of the sensor.

In an embodiment, the control unit further displays, on the display, a two-dimensional image representing a cross section indicated by tomographic data acquired by the sensor at a position corresponding to a position of the element after displacement every time the element is displaced by the element being dragged.

In an embodiment, the control unit colors, using a predetermined color, at least a voxel representing an inside surface of the biological tissue or a voxel adjacent to a voxel representing a corresponding inside surface and representing the lumen in a voxel group at a position corresponding to a position after displacement of the element in a three-dimensional image that is the image every time the element is displaced by the element being dragged.

In an embodiment, the control unit further displays, on the display, a numerical value indicating a movement distance of the sensor corresponding to a distance from a position of the element before dragging to a position of the element after dragging while the element is being dragged.

In an embodiment, the control unit receives, as the user operation, an operation of clicking or tapping a position away from the element by a distance corresponding to a movement distance of the sensor.

In an embodiment, the control unit adjusts a movement speed of the sensor, and causes the sensor to reach a movement destination in a certain period of time regardless of a distance from a position of the element before clicking or tapping to a position of clicking or tapping.

In an embodiment, the control unit combines a first graphic element that is the element and a second graphic element representing a movement range of the sensor and displays the first graphic element and the second graphic element on the display.

In an embodiment, the control unit displays the second graphic element on the display in a direction in which a longitudinal direction of the lumen in a three-dimensional image that is the image is parallel to a longitudinal direction of the second graphic element.

In an embodiment, in a three-dimensional image that is the image, the control unit colors, as the element, at least a voxel representing an inside surface of the biological tissue or a voxel adjacent to a voxel representing a corresponding inside surface and representing the lumen in a first voxel group corresponding to a cross section indicated by tomographic data newly acquired by the sensor separately from a second voxel group corresponding to another cross section of the biological tissue.

An image processing system as one aspect of the present disclosure includes the image processing device, and a probe including the sensor.

In an embodiment, the image processing system further includes the display.

An image display method as one aspect of the present disclosure is an image display method of displaying an image representing a biological tissue on a display and displaying an element representing a position of a sensor on a screen same as the image on the display, based on tomographic data acquired by the sensor moving in a lumen of the biological tissue, the image display method including, by a computer, when a user operation of specifying a movement destination of the sensor is performed on the screen, controlling movement of the sensor according to the user operation, and changing a relative position of the element to cause a position of the sensor after movement to be represented.

A non-transitory computer-readable medium storing an image processing program as one aspect of the present disclosure that causes a computer to display an image representing a biological tissue on a display and to display an element representing a position of a sensor on a screen same as the image on the display, based on tomographic data acquired by the sensor moving in a lumen of the biological tissue by executing a process including, when a user operation of specifying a movement destination of the sensor is performed on the screen, controlling movement of the sensor according to the user operation, and changing a relative position of the element to cause a position of the sensor after movement to be represented.

According to the present disclosure, a sensor can be operated on a screen.

### BRIEF DESCRIPTION OF THE DRAWINGS

FIG. 1 is a perspective view of an image processing system according to an embodiment of the present disclosure.

FIG. 2 is a diagram illustrating an example of a screen displayed on a display by the image processing system according to the embodiment of the present disclosure.

FIG. 3 is a diagram illustrating examples of a two-dimensional image displayed on a display by the image processing system according to the embodiment of the present disclosure.

FIG. 4 is a diagram illustrating an example of a cutting region formed by the image processing system according to the embodiment of the present disclosure.

FIG. 5 is a block diagram illustrating a configuration of an image processing device according to the embodiment of the present disclosure.

FIG. 6 is a diagram illustrating an example of a screen displayed on the display by the image processing system according to a modification of the embodiment of the present disclosure.

FIG. 7 is a perspective view of a probe and a drive unit according to the embodiment of the present disclosure.

FIG. 8 is a flowchart illustrating operation of the image processing system according to the embodiment of the present disclosure.

FIG. 9 is a flowchart illustrating operation of the image processing system according to the embodiment of the present disclosure.

FIG. 10 is a diagram illustrating a result of binarizing a cross-sectional image of a biological tissue in the embodiment of the present disclosure.

FIG. 11 is a diagram illustrating a result of extracting a point cloud of an inside surface of the biological tissue in the embodiment of the present disclosure.

FIG. 12 is a diagram illustrating a result of calculating centroid positions of a cross section of the biological tissue in the embodiment of the present disclosure.

FIG. 13 is a diagram illustrating a result of calculating centroid positions of a plurality of cross sections of the biological tissue in the embodiment of the present disclosure.

FIG. 14 is a diagram illustrating a result of smoothing the result of FIG. 13.

FIG. 15 is a flowchart illustrating operation of the image processing system according to the embodiment of the present disclosure.

FIG. 16 is a diagram illustrating an example of a screen displayed on the display by the image processing system according to a modification of the embodiment of the present disclosure.

### DETAILED DESCRIPTION

Set forth below with reference to the accompanying drawings is a detailed description of embodiments of an image processing device, an image processing system, an image display method, and an image processing program.

In the drawings, the same or corresponding portions are denoted by the same reference numerals. In the description of each of the embodiments, description of the same or corresponding parts will be omitted or simplified as appropriate.

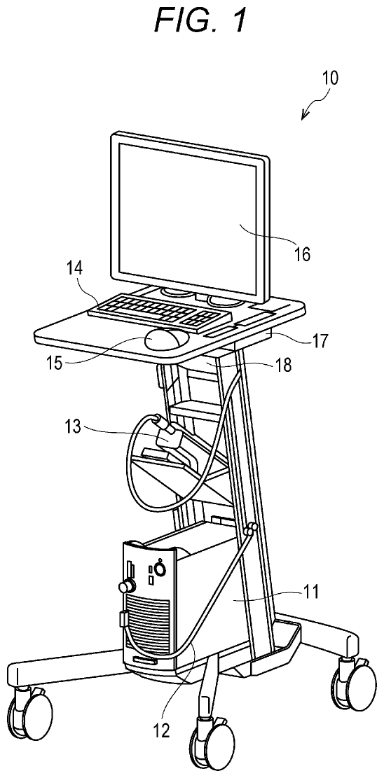

An outline of the present embodiment will be described with reference to FIGS. 1 to 5.

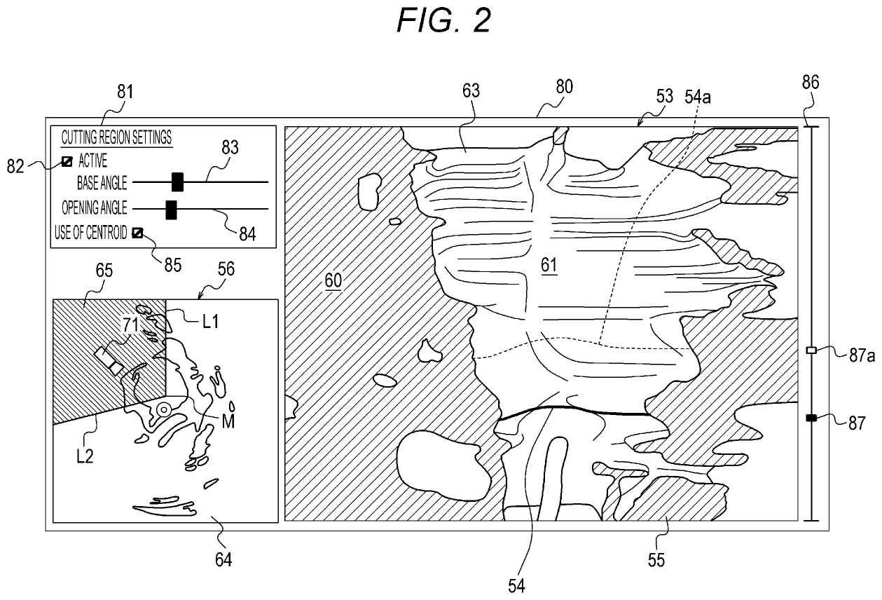

An image processing device 11 according to the present embodiment is a computer that displays an image representing a biological tissue 60 on a display 16 based on tomographic data 51 acquired by a sensor moving in a lumen 63 of the biological tissue 60, and displays an element representing the position of the sensor on a screen 80 same as the image on the display 16. In the present embodiment, the image representing the biological tissue 60 is a three-dimensional image 53 as illustrated on the right part of FIG. 2, but may be a two-dimensional image 56 such as a cross-sectional image as illustrated on the left part of FIG. 2. In the present embodiment, the element representing the position of the sensor is a graphic element, for example, such as a slider knob as illustrated in FIG. 2 as a first graphic element 87, but may be voxels colored using a first color such as green as illustrated in FIG. 2 among a first voxel group 54 corresponding to the position of the sensor. When a user operation of specifying a movement destination of the sensor is performed on the screen 80, the image processing device 11 controls movement of the sensor according to the user operation, and changes the relative position of the element to cause the position of the sensor after the movement to be represented (or displayed).

According to the present embodiment, the sensor can be operated on the screen 80. Therefore, for example, in a case where a procedure such as ablation using IVUS is performed, a doctor performing the procedure while operating a catheter can relatively easily operate an IVUS pullback unit on the screen 80. Alternatively, a clinical technician operating an IVUS system while observing the display 16 can relatively easily operate a pullback unit on the screen 80. Therefore, a procedure can be rather easily performed even in a state where the number of staff members is relatively small.

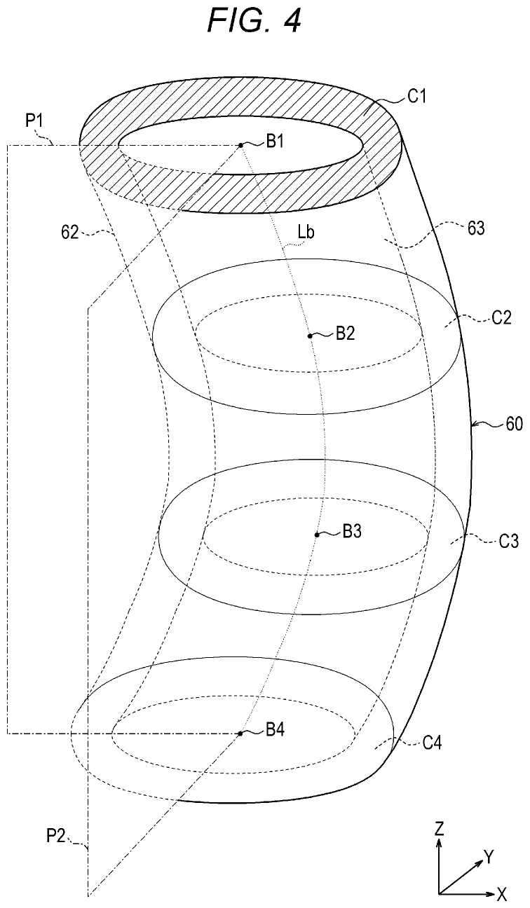

The image processing device 11 displays the three-dimensional data 52 representing the biological tissue 60 as a three-dimensional image 53 on the display 16. As illustrated in FIG. 4, the image processing device 11 forms, in the three-dimensional data 52, a cutting region 62 for exposing the lumen 63 of the biological tissue 60 in the three-dimensional image 53. As illustrated in FIG. 2, the image processing device 11 displays the two-dimensional image 56 representing a cross section 64 of the biological tissue 60 and a region 65 corresponding to the cutting region 62 in the cross section 64 on the display 16 together with the three-dimensional image 53.

According to the present embodiment, how a part of the structure of the biological tissue 60 is cut can be indicated. Therefore, the user can grasp from the two-dimensional image 56 what type of structure the portion of the biological tissue 60 that is cut and which is not displayed in the three-dimensional image 53. For example, in a case where the user is an operator, an operation on the inside of the biological tissue 60 can be relatively easily performed.

The image processing device 11 generates and updates the three-dimensional data 52 based on the tomographic data 51. As illustrated in FIG. 2, in the three-dimensional image 53, the image processing device 11 colors at least voxels representing an inside surface 61 of the biological tissue 60 or voxels adjacent to the voxels representing the inside surface 61 and representing the lumen 63 in the first voxel group 54 corresponding to the cross section 64 indicated by the tomographic data 51 newly acquired by the sensor separately from a second voxel group 55 corresponding to other cross sections of the biological tissue 60.

According to the present embodiment, which portion in the three-dimensional image 53 the cross section 64 of the biological tissue 60 indicated by the tomographic data 51 newly acquired by the sensor corresponds to can be indicated. Therefore, it is rather easy for the user observing the lumen 63 of the biological tissue using the three-dimensional image 53 to grasp which portion in the three-dimensional image 53 information currently obtained by the sensor, that is, the latest information corresponds to.

In a modification of the present embodiment, not only the first voxel group 54, but also at least voxels representing the inside surface 61 or voxels adjacent to the voxels representing the inside surface 61 and representing the lumen 63 in a voxel group corresponding to a cross section adjacent to the cross section 64 to which the first voxel group 54 corresponds may be colored separately from the voxel group corresponding to other cross sections of the biological tissue 60. According to this modification, the width in the movement direction of the sensor of the voxel group colored separately from the voxel group corresponding to other cross sections is widened, and the user can rather easily recognize the voxel group in the three-dimensional image 53.

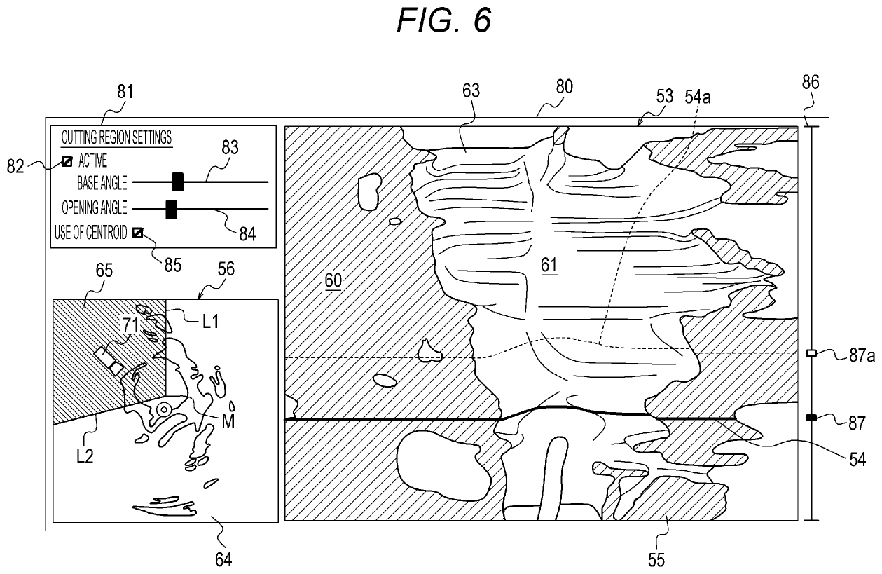

In a modification of the present embodiment, as illustrated in FIG. 6, all voxels representing the biological tissue 60 in the first voxel group 54 may be colored separately from the second voxel group 55. According to this modification, since the first voxel group 54 is colored separately from the second voxel group 55 also on the cutting plane of the biological tissue 60 formed for observing the lumen 63 of the biological tissue 60, it is relatively easier for the user to grasp which portion in the three-dimensional image 53 the latest information corresponds to.

In the present embodiment, the image processing device 11 displays the two-dimensional image 56 representing the cross section 64 on the display 16 together with the three-dimensional image 53 in which at least voxels representing the inside surface 61 of the biological tissue 60 or voxels adjacent to the voxels representing the inside surface 61 and representing the lumen 63 in the first voxel group 54 corresponding to the cross section 64 are colored separately from the second voxel group 55 corresponding to other cross sections. Therefore, the relationship between the two-dimensional image 56 and the three-dimensional image 53 can be indicated.

The biological tissue 60 can include, for example, an organ such as a blood vessel or a heart. The biological tissue 60 is not limited to only an anatomically single organ or a part of the anatomically single organ, but also includes a tissue having a lumen across a plurality of organs. An example of such a tissue can be, for example, specifically, a part of the vascular tissue extending from the upper part of the inferior vena cava to the lower part of the superior vena cava through the right atrium. In the examples of FIGS. 2 to 4, the biological tissue 60 is a blood vessel.

In FIG. 2, an operation panel 81, the two-dimensional image 56, the three-dimensional image 53, the first graphic element 87, and a second graphic element 86 are displayed on the screen 80.

The operation panel 81 is a graphical user interface (GUI) component for setting the cutting region 62. The operation panel 81 includes a check box 82 for selecting whether to activate settings of the cutting region 62, a slider 83 for setting the base angle, a slider 84 for setting the opening angle, and a check box 85 for selecting whether to use a centroid.

The base angle is a rotation angle of one straight line L1 of two straight lines L1 and L2 extending from one point M in the cross-sectional image representing the cross section 64 of the biological tissue 60. Therefore, setting the base angle corresponds to setting the direction of the straight line L1. The opening angle is an angle between the two straight lines L1 and L2. Therefore, setting the opening angle corresponds to setting the angle formed by the two straight lines L1 and L2. The point M is the centroid of the cross section 64. In a case where non-use of a centroid is selected, the point M may be set to a point other than the centroid on the cross section 64.

The two-dimensional image 56 is an image obtained by processing a cross-sectional image. In the two-dimensional image 56, the color of the region 65 corresponding to the cutting region 62 is changed in order to clearly indicate which portion of the cross section 64 is cut.

In the present embodiment, the viewpoint when displaying the three-dimensional image 53 on the screen 80 can be adjusted according to the position of the cutting region 62. The viewpoint is a position of a virtual camera 71 arranged in a three-dimensional space. In the two-dimensional image 56, the position of the camera 71 with respect to the cross section 64 is displayed.

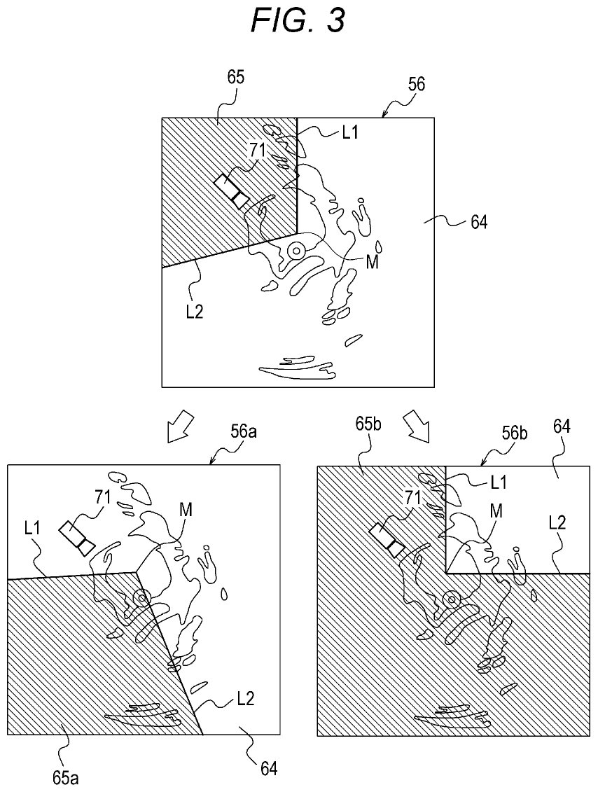

In the present embodiment, the cutting region 62 can be determined using the two-dimensional image 56. Specifically, as illustrated in FIG. 3, the position or size of the cutting region 62 can be set by adjusting the base angle or the opening angle to set the position or size of the region 65 obtained by dividing by the two straight lines L1 and L2 in the two-dimensional image 56. For example, in a case where the base angle is changed such that the straight line L1 rotates counterclockwise by about 90 degrees, a region 65a moved according to the change of the base angle is obtained in a two-dimensional image 56a. Then, the position of the cutting region 62 can be adjusted according to the position of the region 65a. Alternatively, in a case where the opening angle is changed such that the angle between the two straight lines L1 and L2 is increased, a region 65b enlarged according to the change in the opening angle is obtained in a two-dimensional image 56b. Then, the size of the cutting region 62 is adjusted according to the size of the region 65b. Both the position and the size of the cutting region 62 can be set by adjusting both the base angle and the opening angle to set both the position and the size of the region 65 in the two-dimensional image 56. The position of the camera 71 may be appropriately adjusted according to the position or size of the cutting region 62.

In the present embodiment, an image corresponding to the current position of the sensor, that is, the latest image is always displayed as the two-dimensional image 56, but in a modification of the present embodiment, an image corresponding to a position other than the current position of the sensor may be displayed as the two-dimensional image 56 after the cutting region 62 is determined.

In a modification of the present embodiment, the base angle may be set by dragging the straight line L1 or by inputting a numerical value instead of being set by operating the slider 83. Similarly, the opening angle may be set by dragging the straight line L2 or by inputting a numerical value instead of being set by operating the slider 84.

In the three-dimensional image 53, the cutting region 62 determined using the two-dimensional image 56 is hidden or transparent. In the three-dimensional image 53, the sensor is currently present in the longitudinal direction of the lumen 63, and the color of the first voxel group 54 corresponding to the current position of the sensor is changed in order to express the position where update is currently performed in real time.

In the present embodiment, as illustrated in FIG. 2, voxels representing the inside surface 61 of the biological tissue 60 in the first voxel group 54 are set to a color different from that of the second voxel group 55, so that the voxels are colored separately from the second voxel group 55. However, in a modification of the present embodiment, as illustrated in FIG. 6, all voxels representing the biological tissue 60 in the first voxel group 54 may be set to a different color. In a further modification, instead of setting the first voxel group 54 and the second voxel group 55 to different colors, the contrast between the first voxel group 54 and the second voxel group 55 is adjusted, so that the first voxel group 54 may be colored separately from the second voxel group 55.

The first graphic element 87 is a graphic element representing the position of the sensor. The second graphic element 86 is a graphic element representing a range of movement of the sensor. In the present embodiment, a combination of the first graphic element 87 and the second graphic element 86 is configured as a slider. The first graphic element 87 and the second graphic element 86 may be displayed at any position, but are displayed on the right part of the three-dimensional image 53 in the present embodiment.

In the present embodiment, when an operation of dragging the first graphic element 87 and dropping the first graphic element 87 at the position corresponding to the movement destination of the sensor is performed as a user operation, the movement of the sensor is controlled and the relative position of the first graphic element 87 is changed according to the user operation. For example, assuming that no user operation is currently performed, as illustrated in FIG. 2, voxels representing the inside surface 61 of the biological tissue 60 in the first voxel group 54 corresponding to the current position of the sensor are colored using a first color such as green. The first graphic element 87 represents the current position of the sensor by being located at the same height as the first voxel group 54. Assuming that an operation of dragging the first graphic element 87 upward and dropping the first graphic element 87 at a certain position is performed thereafter as a user operation, the sensor automatically moves to a position corresponding to the position. Then, voxels representing the inside surface 61 of the biological tissue 60 in the first voxel group 54a corresponding to the position of the sensor after movement are colored using the first color such as green. A dropped first graphic element 87a represents the position of the sensor after the movement by being located at the same height as the drop destination, that is, the first voxel group 54a.

In a modification of the present embodiment, the user operation may be a click or tap operation instead of a drag and drop operation. That is, as the user operation, an operation of clicking or tapping a position away from the first graphic element 87 by a distance corresponding to the movement distance of the sensor may be performed. For example, assuming that no user operation is currently performed, as illustrated in FIG. 2, voxels representing the inside surface 61 of the biological tissue 60 in the first voxel group 54 corresponding to the current position of the sensor are colored using a first color such as green. The first graphic element 87 represents the current position of the sensor by being located at the same height as the first voxel group 54. Assuming that an operation of clicking or tapping a position away upward from the first graphic element 87 is performed thereafter as a user operation, the sensor automatically moves to a position corresponding to the position. Then, voxels representing the inside surface 61 of the biological tissue 60 in the first voxel group 54a corresponding to the position of the sensor after movement are colored using the first color such as green. The dropped first graphic element 87a representing the position of the sensor after the movement is displayed at the clicked or tapped position by being located at the same height as the first voxel group 54a. In order to prevent an erroneous operation, the user operation may include an operation of pressing a specific key such as a shift key simultaneously with a click or tap operation.

In a modification of the present embodiment, instead of dragging and dropping the first graphic element 87, the voxels colored using the first color such as green in the first voxel group 54 may be dragged and dropped. In that case, the voxels colored using the first color can be regarded as a line of the first color. That is, when an operation of dragging the line of the first color such as green and dropping the line of the first color at the position corresponding to the movement destination of the sensor is performed as a user operation, the movement of the sensor is controlled and the relative position of the line of the first color is changed according to the user operation. For example, assuming that no user operation is currently performed, as illustrated in FIG. 2, voxels representing the inside surface 61 of the biological tissue 60 in the first voxel group 54 corresponding to the current position of the sensor are colored using the first color such as green, and function as a line representing the current position of the sensor. Assuming that an operation of dragging the line upward and dropping the line at a certain position is performed thereafter as a user operation, the sensor automatically moves to a position corresponding to the position. Then, voxels representing the inside surface 61 of the biological tissue 60 in the first voxel group 54a corresponding to the position of the sensor after movement are colored using the first color such as green, and function as a line representing the position of the sensor after the movement.

In a further modification of this modification, the user operation may be a simple click or tap operation instead of a drag and drop operation. That is, as the user operation, an operation of clicking or tapping a position away from the line of the first color such as green by a distance corresponding to the movement distance of the sensor may be performed. For example, assuming that no user operation is currently performed, as illustrated in FIG. 2, voxels representing the inside surface 61 of the biological tissue 60 in the first voxel group 54 corresponding to the current position of the sensor are colored using the first color such as green, and function as a line representing the current position of the sensor. Assuming that an operation of clicking or tapping a position away upward from the line is performed thereafter as a user operation, the sensor automatically moves to a position corresponding to the position. Then, voxels representing the inside surface 61 of the biological tissue 60 in the first voxel group 54a corresponding to the position of the sensor after movement are colored using the first color such as green, and function as a line representing the position of the sensor after the movement. In order to prevent an erroneous operation, the user operation may include an operation of pressing a specific key such as a shift key simultaneously with a click or tap operation.

In FIG. 4, the X direction, and the Y direction orthogonal to the X direction correspond to the lateral directions of the lumen 63 of the biological tissue 60. The Z direction orthogonal to the X direction and the Y direction corresponds to the longitudinal direction of the lumen 63 of the biological tissue 60.

In the example of FIG. 4, it is assumed that the check box 85 of the operation panel 81 is in a checked state, that is, use of a centroid is selected. The image processing device 11 calculates the positions of centroids B1, B2, B3, and B4 of cross sections C1, C2, C3, and C4 of the biological tissue 60 using the three-dimensional data 52. The image processing device 11 sets two planes intersecting at a single line Lb passing through the positions of the centroids B1, B2, B3, and B4 and including the two respective straight lines L1 and L2 as cutting planes P1 and P2. For example, assuming that the point M illustrated in FIG. 2 is the point B3, the straight line L1 is an intersection line between the cross section C3 and the cutting plane P1, and the straight line L2 is an intersection line between the cross section C3 and the cutting plane P2. The image processing device 11 forms, in the three-dimensional data 52, a region interposed between the cutting planes P1 and P2 in the three-dimensional image 53 and from which the lumen 63 of the biological tissue 60 is exposed, as the cutting region 62.

In the case of the three-dimensional model of the bent blood vessel as illustrated in FIG. 4, there is a case where the inside of the blood vessel cannot be correctly displayed if the three-dimensional model is cut at one plane to display the lumen 63. In the present embodiment, as illustrated in FIG. 4, by continuously capturing the centroids of the blood vessel, the three-dimensional model can be cut such that the inside of the blood vessel can be reliably displayed.

In FIG. 4, for convenience, the four cross sections C1, C2, C3, and C4 are illustrated as a plurality of lateral cross sections of the lumen 63 of the biological tissue 60, but the number of cross sections serving as calculation targets of the centroid positions is not limited to four, and is preferably the same as the number of cross-sectional images acquired by IVUS.

In another example different from FIG. 4, it is assumed that the check box 85 of the operation panel 81 is not in a checked state, that is, non-use of a centroid is selected. In such an example, the image processing device 11 sets two planes intersecting at any one line passing through the point M, such as a straight line extending in the Z direction through the point M, and including the respective two straight lines L1 and L2, as the cutting planes P1 and P2.

A configuration of an image processing system 10 according to the present embodiment will be described with reference to FIG. 1.

The image processing system 10 includes the image processing device 11, a cable 12, a drive unit 13, a keyboard 14, a mouse 15, and the display 16.

In the present embodiment, the image processing device 11 is a dedicated computer specialized for image diagnosis, but may be a general-purpose computer such as a personal computer (PC).

The cable 12 is used to connect the image processing device 11 and the drive unit 13.

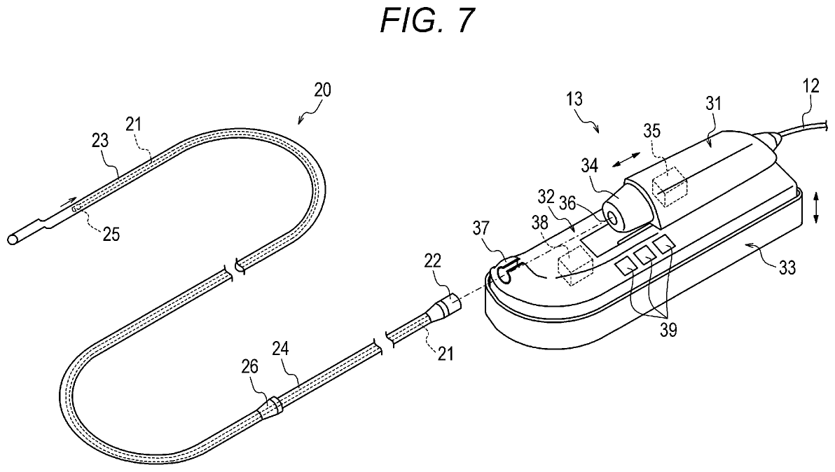

The drive unit 13 is a device that is used by being connected to a probe 20 illustrated in FIG. 7 and drives the probe 20. The drive unit 13 is also referred to as a motor drive unit (MDU). The probe 20 is applied to IVUS. The probe 20 is also called an IVUS catheter or an image diagnosis catheter.

The keyboard 14, the mouse 15, and the display 16 are connected to the image processing device 11 via any cable or wirelessly. The display 16 is, for example, a liquid crystal display (LCD), an organic electro luminescence (EL) display, or a head-mounted display (HMD).

The image processing system 10 optionally further includes a connection terminal 17 and a cart unit 18.

The connection terminal 17 is used to connect the image processing device 11 and an external device. The connection terminal 17 is, for example, a universal serial bus (USB) terminal. The external device can be, for example, a recording medium such as a magnetic disk drive, a magneto-optical disk drive, or an optical disk drive.

The cart unit 18 can be, for example, a cart with a caster for movement. The image processing device 11, the cable 12, and the drive unit 13 can be, for example, installed in the cart body of the cart unit 18. The keyboard 14, the mouse 15, and the display 16 can be, for example, installed on the uppermost table of the cart unit 18.

Configurations of the probe 20 and the drive unit 13 according to the present embodiment will be described with reference to FIG. 7.

The probe 20 includes a drive shaft 21, a hub 22, a sheath 23, an outer tube 24, an ultrasound transducer 25, and a relay connector 26.

The drive shaft 21 passes through the sheath 23 inserted into the body cavity of the living body and the outer tube 24 connected to the proximal end of the sheath 23, and extends to the inside of the hub 22 disposed at the proximal end of the probe 20. The drive shaft 21 is rotatably disposed in the sheath 23 and the outer tube 24 with an ultrasound transducer 25 that transmits and receives a signal at the distal end. The relay connector 26 connects the sheath 23 and the outer tube 24.

The hub 22, the drive shaft 21, and the ultrasound transducer 25 are connected to each other to integrally move forward and backward in the axial direction. Therefore, for example, when the hub 22 is pushed toward the distal end, the drive shaft 21 and the ultrasound transducer 25 move toward the distal end inside the sheath 23. For example, when the hub 22 is pulled toward the proximal end, the drive shaft 21 and the ultrasound transducer 25 move toward the proximal end inside the sheath 23 as indicated by arrows.

The drive unit 13 includes a scanner unit 31, a slide unit 32, and a bottom cover 33.

The scanner unit 31 is also referred to as a pullback unit. The scanner unit 31 is connected to the image processing device 11 via the cable 12. The scanner unit 31 includes a probe connection portion 34 connected to the probe 20 and a scanner motor 35 as a drive source for rotating the drive shaft 21.

The probe connection portion 34 is detachably connected to the probe 20 via an insertion port 36 of the hub 22 disposed at the proximal end of the probe 20. In the hub 22, the proximal end of the drive shaft 21 is rotatably supported, and the rotational force of the scanner motor 35 is transmitted to the drive shaft 21. In addition, signals are transmitted and received between the drive shaft 21 and the image processing device 11 via the cable 12. In the image processing device 11, generation of a tomographic image of a biological lumen and image processing are performed based on a signal transmitted from the drive shaft 21.

The slide unit 32 is mounted with the scanner unit 31 to be movable forward and backward, and is mechanically and electrically connected to the scanner unit 31. The slide unit 32 includes a probe clamp portion 37, a slide motor 38, and a switch group 39.

The probe clamp portion 37 is disposed coaxially with the probe connection portion 34 at a position distal of the probe connection portion 34, and supports the probe 20 connected to the probe connection portion 34.

The slide motor 38 is a drive source that generates a drive force in the axial direction. The scanner unit 31 moves forward and backward by the drive of the slide motor 38, and the drive shaft 21 moves forward and backward in the axial direction accordingly. The slide motor 38 is, for example, a servo motor.

The switch group 39 can include, for example, a forward switch and a pull-back switch that are pressed at the time of the forward and backward operation of the scanner unit 31, and a scan switch that is pressed at the time of the start and end of image depiction. The present disclosure is not limited to the examples described herein, and various switches are included in the switch group 39, as necessary.

When the forward switch is pressed, the slide motor 38 rotates forward, and the scanner unit 31 moves forward. On the other hand, when the pull-back switch is pressed, the slide motor 38 reversely rotates, and the scanner unit 31 moves backward.

When the scan switch is pressed, image depiction is started, the scanner motor 35 is driven, and the slide motor 38 is driven to move the scanner unit 31 backward. A user such as an operator connects the probe 20 to the scanner unit 31 in advance, and causes the drive shaft 21 to move toward the proximal end in the axial direction while rotating at the start of image depiction. When the scan switch is pressed again, the scanner motor 35 and the slide motor 38 are stopped, and image depiction ends.

The bottom cover 33 covers the entire periphery of the bottom surface and the side surface on the bottom surface side of the slide unit 32, and the bottom cover 33 can be attached to and separate from the bottom surface of the slide unit 32.

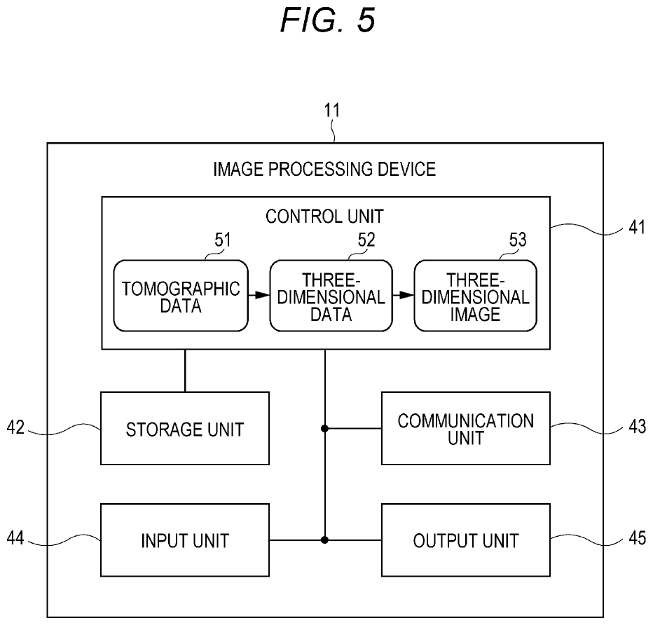

A configuration of the image processing device 11 will be described with reference to FIG. 5.

The image processing device 11 includes a control unit 41, a storage unit 42, a communication unit 43, an input unit 44, and an output unit 45.

The control unit 41 includes at least one processor, at least one programmable circuit, at least one dedicated circuit, or any combination of the at least one processor, the at least one programmable circuit, and the at least one dedicated circuit. The processor can be, for example, a general-purpose processor such as a central processing unit (CPU) or a graphics processing unit (GPU), or a dedicated processor specialized for specific processing. The programmable circuit can be, for example, field-programmable gate array (FPGA). The dedicated circuit can be, for example, an application specific integrated circuit (ASIC). The control unit 41 executes processing related to the operation of the image processing device 11 while controlling each unit of the image processing system 10 including the image processing device 11.

The storage unit 42 includes at least one semiconductor memory, at least one magnetic memory, at least one optical memory, or any combination of the at least one semiconductor memory, the at least one magnetic memory, and the at least one optical memory. The semiconductor memory can be, for example, a random access memory (RAM) or a read only memory (ROM). The RAM can be, for example, a static random access memory (SRAM) or a dynamic random access memory (DRAM). The ROM can be, for example, an electrically erasable programmable read only memory (EEPROM). The storage unit 42 can function as, for example, a main storage device, an auxiliary storage device, or a cache memory. The storage unit 42 stores data to be used for the operation of the image processing device 11 such as the tomographic data 51 and data obtained by the operation of the image processing device 11 such as the three-dimensional data 52 and the three-dimensional image 53.

The communication unit 43 includes at least one communication interface. The communication interface can be, for example, a wired LAN interface, a wireless local area network (LAN) interface, or an image diagnosis interface that receives and analog to digital (ND) converts an IVUS signal. The communication unit 43 receives data to be used for the operation of the image processing device 11 and transmits data obtained by the operation of the image processing device 11. In the present embodiment, the drive unit 13 is connected to the image diagnosis interface included in the communication unit 43.

The input unit 44 includes at least one input interface. The input interface can be, for example, a USB interface, a high-definition multimedia interface (HDMI®) interface, or an interface compatible with near-field communication standard, such as Bluetooth®. The input unit 44 receives a user's operation such as an operation of inputting data to be used for the operation of the image processing device 11. In the present embodiment, the keyboard 14 and the mouse 15 are connected to a USB interface or an interface compatible with near-field communication included in the input unit 44. In a case where the touch screen is disposed integrally with the display 16, the display 16 may be connected to the USB interface or the HDMI interface included in the input unit 44.

The output unit 45 includes at least one output interface. The output interface can be, for example, a USB interface, an HDMI interface, or an interface compatible with near-field communication standard, such as Bluetooth. The output unit 45 outputs data obtained by the operation of the image processing device 11. In the present embodiment, the display 16 is connected to a USB interface or an HDMI interface included in the output unit 45.

A function of the image processing device 11 is implemented by executing an image processing program according to the present embodiment by a processor serving as the control unit 41. That is, the function of the image processing device 11 is implemented by software. The image processing program causes a computer to execute the operation of the image processing device 11 to cause the computer to function as the image processing device 11. That is, the computer functions as the image processing device 11 by executing the operation of the image processing device 11 according to the image processing program.

The program can be stored in a non-transitory computer-readable medium. The non-transitory computer-readable medium can be, for example, a flash memory, a magnetic recording device, an optical disc, a magneto-optical recording medium, or a ROM. The program is distributed, for example, by selling, transferring, or lending a portable medium, such as a secure digital (SD) card, a digital versatile disc (DVD), or a compact disc read only memory (CD-ROM), storing the program. The program may be distributed by storing the program in a storage of a server and transferring the program from the server to another computer. The program may be provided as a program product.

The computer can temporarily store, for example, a program stored in a portable medium or a program transferred from the server in the main storage. Then, the computer reads the program stored in the main storage by the processor and executes processing according to the read program by the processor. The computer may read the program directly from the portable medium and execute the processing according to the program. Each time the program is transferred from a server to a computer, the computer may sequentially execute processing according to the received program. The processing may be executed by what is called an application service provider (ASP) service in which the functions are implemented only by execution instructions and result acquisition instead of the program being transferred from the server to the computer. The program includes information that is provided for processing by an electronic computer and is equivalent to the program. For example, data that is not a direct command to the computer but has a property that defines processing of the computer corresponds to the “information equivalent to the program”.

Some or all of the functions of the image processing device 11 may be implemented by a programmable circuit or a dedicated circuit as the control unit 41. That is, some or all of the functions of the image processing device 11 may be implemented by hardware.

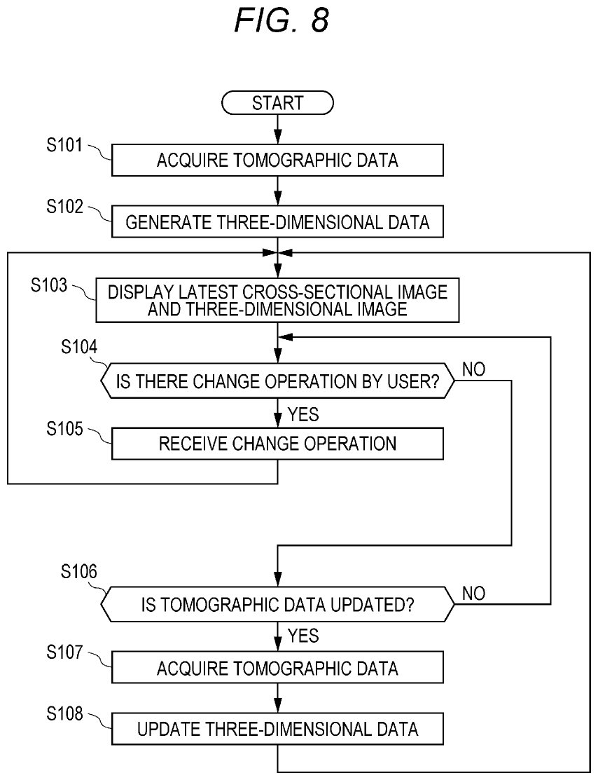

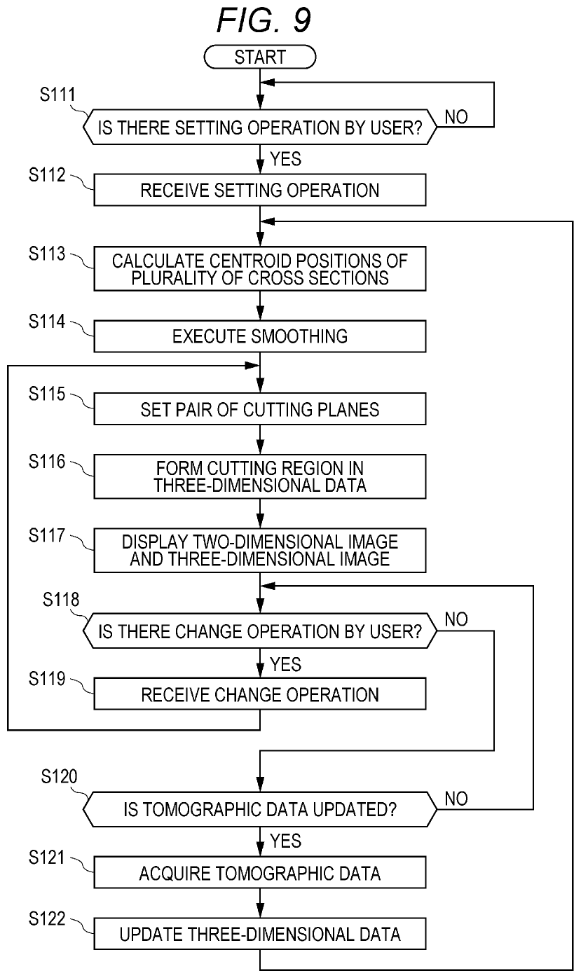

The operation of the image processing system 10 according to the present embodiment will be described with reference to FIGS. 8 and 9. The operation of the image processing system 10 corresponds to an image display method according to the present embodiment.

Before a start of a flow of FIG. 8, the probe 20 is primed by the user. Thereafter, the probe 20 is fitted into the probe connection portion 34 and the probe clamp portion 37 of the drive unit 13, and is connected and fixed to the drive unit 13. Then, the probe 20 is inserted to a target site in the biological tissue 60 such as a blood vessel or the heart.

In S101, the scan switch included in the switch group 39 is pressed, and the pull-back switch included in the switch group 39 is further pressed, so that a so-called pull-back operation is performed. The probe 20 transmits an ultrasonic wave inside the biological tissue 60 by the ultrasound transducer 25 that moves backward in the axial direction by the pull-back operation. The ultrasound transducer 25 radially transmits the ultrasound wave while moving inside the biological tissue 60. The ultrasound transducer 25 receives a reflected wave of the transmitted ultrasound wave. The probe 20 inputs a signal of the reflected wave received by the ultrasound transducer 25 to the image processing device 11. The control unit 41 of the image processing device 11 processes the input signal to sequentially generate cross-sectional images of the biological tissue 60, thereby acquiring the tomographic data 51, which includes a plurality of cross-sectional images.

Specifically, the probe 20 transmits the ultrasonic wave in a plurality of directions from a rotation center to the outside by the ultrasound transducer 25 while rotating the ultrasound transducer 25 in the circumferential direction and moving the ultrasound transducer 25 in the axial direction inside the biological tissue 60. The probe 20 receives the reflected wave from a reflecting object existing in each of a plurality of directions inside the biological tissue 60 by the ultrasound transducer 25. The probe 20 transmits the signal of the received reflected wave to the image processing device 11 via the drive unit 13 and the cable 12. The communication unit 43 of the image processing device 11 receives the signal transmitted from the probe 20. The communication unit 43 performs ND conversion on the received signal. The communication unit 43 inputs the ND converted signal to the control unit 41. The control unit 41 processes the input signal to calculate an intensity value distribution of the reflected wave from the reflecting object existing in the transmission direction of the ultrasonic wave of the ultrasound transducer 25. The control unit 41 sequentially generates two-dimensional images having a luminance value distribution corresponding to the calculated intensity value distribution as the cross-sectional images of the biological tissue 60, thereby acquiring tomographic data 51, which is a data set of the cross-sectional images. The control unit 41 stores the acquired tomographic data 51 in the storage unit 42.

In the present embodiment, the signal of the reflected wave received by the ultrasound transducer 25 corresponds to raw data of the tomographic data 51, and the cross-sectional images generated by processing the signal of the reflected wave by the image processing device 11 correspond to processed data of the tomographic data 51.

In a modification of the present embodiment, the control unit 41 of the image processing device 11 may store the signal input from the probe 20 as it is in the storage unit 42 as the tomographic data 51. Alternatively, the control unit 41 may store data indicating the intensity value distribution of the reflected wave calculated by processing the signal input from the probe 20 in the storage unit 42 as the tomographic data 51. That is, the tomographic data 51 is not limited to the data set of the cross-sectional images of the biological tissue 60, and may be data representing a cross section of the biological tissue 60 at each movement position of the ultrasound transducer 25 in any format.

In a modification of the present embodiment, an ultrasound transducer that transmits the ultrasound wave in the plurality of directions without rotating may be used instead of the ultrasound transducer 25 that transmits the ultrasound wave in the plurality of directions while rotating in the circumferential direction.

In a modification of the present embodiment, the tomographic data 51 may be acquired using optical frequency domain imaging (OFDI) or optical coherence tomography (OCT) instead of being acquired using IVUS. In a case where OFDI or OCT is used, as a sensor that acquires the tomographic data 51 while moving in the lumen 63 of the biological tissue 60, a sensor that acquires the tomographic data 51 by emitting light in the lumen 63 of the biological tissue 60 is used instead of the ultrasound transducer 25 that acquires the tomographic data 51 by transmitting the ultrasound wave in the lumen 63 of the biological tissue 60.

In a modification of the present embodiment, instead of the image processing device 11 generating the data set of the cross-sectional images of the biological tissue 60, another device may generate a similar data set, and the image processing device 11 may acquire the data set from the another device. That is, instead of the control unit 41 of the image processing device 11 processing the IVUS signal to generate the cross-sectional images of the biological tissue 60, another device may process the IVUS signal to generate the cross-sectional images of the biological tissue 60 and input the generated cross-sectional images to the image processing device 11.

In S102, the control unit 41 of the image processing device 11 generates the three-dimensional data 52 of the biological tissue 60 based on the tomographic data 51 acquired in S101. That is, the control unit 41 generates the three-dimensional data 52 based on the tomographic data 51 acquired by the sensor. Note that at this time, if already generated three-dimensional data 52 is present, it is preferable to update only data at a location corresponding to the updated tomographic data 51, instead of regenerating all the three-dimensional data 52 from the beginning. Accordingly, a data processing amount when generating the three-dimensional data 52 can be reduced, and a real-time property of the three-dimensional image 53 in the subsequent S103 can be improved.

Specifically, the control unit 41 of the image processing device 11 generates the three-dimensional data 52 of the biological tissue 60 by layering the cross-sectional images of the biological tissue 60 included in the tomographic data 51 stored in the storage unit 42 and converting the same into three-dimensional data. As a method of three-dimensional conversion, any method among rendering methods such as surface rendering or volume rendering, and various types of processing associated with the rendering method such as texture mapping including environment mapping and bump mapping can be used. The control unit 41 stores the generated three-dimensional data 52 in the storage unit 42.

In S103, the control unit 41 of the image processing device 11 displays the three-dimensional data 52 generated in S102 on the display 16, as the three-dimensional image 53. The control unit 41 may set an angle for displaying the three-dimensional image 53 to any angle. The control unit 41 displays the latest cross-sectional image included in the tomographic data 51 acquired in S101 on the display 16 together with the three-dimensional image 53.

Specifically, the control unit 41 of the image processing device 11 generates the three-dimensional image 53 based on the three-dimensional data 52 stored in the storage unit 42. The control unit 41 displays the latest cross-sectional image among the cross-sectional images of the biological tissue 60 included in the tomographic data 51 stored in the storage unit 42 and the generated three-dimensional image 53 on the display 16 via the output unit 45.

In the present embodiment, in the three-dimensional image 53, the control unit 41 of the image processing device 11 colors voxels representing the inside surface 61 of the biological tissue 60 in the first voxel group 54 corresponding to the cross section 64 indicated by the tomographic data 51 newly acquired by the sensor separately from the second voxel group 55 corresponding to other cross sections of the biological tissue 60. Specifically, as illustrated in FIG. 2, the control unit 41 sets the color of the voxels representing the inside surface 61 of the biological tissue 60 in the first voxel group 54 to a color different from any color of the second voxel group 55, thereby coloring the voxels representing the inside surface 61 of the biological tissue 60 in the first voxel group 54 separately from the second voxel group 55.

In a modification of the present embodiment, as illustrated in FIG. 6, the control unit 41 of the image processing device 11 may color all the voxels representing the biological tissue 60 in the first voxel group 54 separately from the second voxel group 55. Specifically, the control unit 41 may set the color of all the voxels representing the biological tissue 60 in the first voxel group 54 to a color different from any color of the second voxel group 55, thereby coloring all the voxels representing the biological tissue 60 in the first voxel group 54 separately from the second voxel group 55.

In the present embodiment, the control unit 41 of the image processing device 11 combines the first graphic element 87 and the second graphic element 86 and displays the first graphic element 87 and the second graphic element 86 on the display 16 together with the three-dimensional image 53. Specifically, as illustrated in FIG. 2, the control unit 41 displays a slider configured by combining the first graphic element 87 and the second graphic element 86 on the right part of the three-dimensional image 53 via the output unit 45.

In the present embodiment, the control unit 41 of the image processing device 11 displays the second graphic element 86 on the display 16 in a direction in which the longitudinal direction of the lumen 63 in the three-dimensional image 53 is parallel to the longitudinal direction of the second graphic element 86. Specifically, as illustrated in FIG. 2, the control unit 41 matches a movement range of the sensor indicated by the second graphic element 86 with a display range of the three-dimensional image 53 in the vertical direction of the screen 80, and matches the position of the sensor indicated by the first graphic element 87 with the position of the first voxel group 54.

In S104, if there is an operation of setting the angle for displaying the three-dimensional image 53 as a change operation by the user, processing of S105 is executed. If there is no change operation by the user, processing of S106 is executed.

In S105, the control unit 41 of the image processing device 11 receives, via the input unit 44, the operation of setting the angle for displaying the three-dimensional image 53. The control unit 41 adjusts the angle for displaying the three-dimensional image 53 to the set angle. In S103, the control unit 41 displays the three-dimensional image 53 on the display 16 at the angle set in S105.

Specifically, the control unit 41 of the image processing device 11 receives, via the input unit 44, an operation by the user of rotating the three-dimensional image 53 displayed on the display 16 by using the keyboard 14, the mouse 15, or the touch screen disposed integrally with the display 16. The control unit 41 can interactively adjust the angle for displaying the three-dimensional image 53 on the display 16 according to the operation by the user. Alternatively, the control unit 41 receives, via the input unit 44, an operation by the user of inputting a numerical value of the angle for displaying the three-dimensional image 53 by using the keyboard 14, the mouse 15, or the touch screen disposed integrally with the display 16. The control unit 41 adjusts the angle for displaying the three-dimensional image 53 on the display 16 in accordance with the input numerical value.

If the tomographic data 51 is updated in S106, the processing in S107 and S108 is executed. If the tomographic data 51 is not updated, the presence or absence of the change operation by the user is confirmed again in S104.

In S107, similarly to the processing in S101, the control unit 41 of the image processing device 11 processes the signal input from the probe 20 to newly generate cross-sectional images of the biological tissue 60, thereby acquiring the tomographic data 51 including at least one new cross-sectional image.

In S108, the control unit 41 of the image processing device 11 updates the three-dimensional data 52 of the biological tissue 60 based on the tomographic data 51 acquired in S107. That is, the control unit 41 updates the three-dimensional data 52 based on the tomographic data 51 acquired by the sensor. Then, in S103, the control unit 41 displays the three-dimensional data 52 updated in S108 on the display 16, as the three-dimensional image 53. The control unit 41 displays the latest cross-sectional image included in the tomographic data 51 acquired in S107 on the display 16 together with the three-dimensional image 53. In S108, it can be preferable to update only data at a location corresponding to the updated tomographic data 51. Accordingly, the data processing amount when generating the three-dimensional data 52 can be reduced, and the real-time property of the three-dimensional image 53 can be improved in S108.

In S111, if there is an operation of setting the cutting region 62 as a setting operation by the user, the processing of S112 is executed.

In S112, the control unit 41 of the image processing device 11 receives, via the input unit 44, the operation of setting the cutting region 62.

Specifically, the control unit 41 of the image processing device 11 receives an operation of setting the region 65 corresponding to the cutting region 62 with respect to the cross-sectional image displayed on the display 16 in S103 via the input unit 44. In the present embodiment, the control unit 41 receives an operation of setting the two straight lines L1 and L2 extending from one point M in the cross-sectional image as the operation of setting the region 65 corresponding to the cutting region 62.

More specifically, the control unit 41 of the image processing device 11 receives, via the input unit 44, an operation by the user of specifying the base angle and the opening angle on the operation panel 81 as illustrated in FIG. 2 using the keyboard 14, the mouse 15, or the touch screen disposed integrally with the display 16. That is, as the operation of setting the two straight lines L1 and L2, the control unit 41 receives an operation of specifying the direction of one straight line L1 of the two straight lines L1 and L2 and the angle formed by the two straight lines L1 and L2. Here, it is assumed that the check box 85 of the operation panel 81 is in a checked state, that is, use of a centroid is selected.

In a modification of the present embodiment, the control unit 41 of the image processing device 11 may receive, via the input unit 44, an operation by the user of drawing the two straight lines L1 and L2 on a cross-sectional image displayed on the display 16 by using the keyboard 14, the mouse 15, or the touch screen disposed integrally with the display 16. That is, the control unit 41 may receive an operation of drawing the two straight lines L1 and L2 on the cross-sectional image as the operation of setting the two straight lines L1 and L2.

In S113, the control unit 41 of the image processing device 11 calculates the centroid positions of the plurality of lateral cross sections of the lumen 63 of the biological tissue 60 by using the latest three-dimensional data 52 stored in the storage unit 42. The latest three-dimensional data 52 is the three-dimensional data 52 generated in S102 if the processing in S108 is not executed, and is the three-dimensional data 52 updated in S108 if the processing in S108 is executed. Note that at this time, if already generated three-dimensional data 52 is present, it can be preferable to update only data at a location corresponding to the updated tomographic data 51, instead of regenerating all of the three-dimensional data 52 from the beginning. Accordingly, a data processing amount when generating the three-dimensional data 52 can be reduced, and a real-time property of the three-dimensional image 53 in the subsequent S117 can be improved.

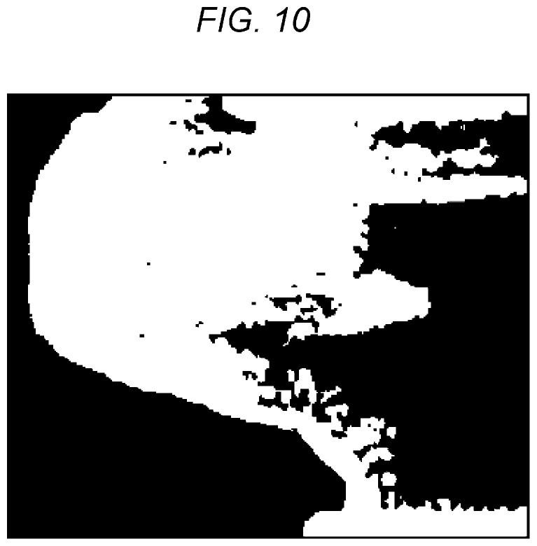

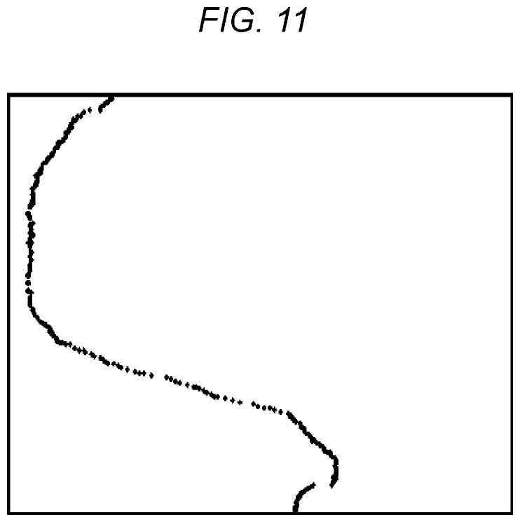

Specifically, as illustrated in FIG. 10, if the control unit 41 of the image processing device 11 generates a corresponding new cross-sectional image in S107 for each of the plurality of cross-sectional images generated in S101, the control unit 41 replaces each of the plurality of cross-sectional images generated in S101 with the new cross-sectional image, and then binarizes the cross-sectional image. As illustrated in FIG. 11, the control unit 41 extracts a point cloud on the inside surface of the biological tissue 60 from the binarized cross-sectional image. For example, the control unit 41 extracts a point cloud on an inside surface of a blood vessel by extracting points corresponding to an inside surface of a main blood vessel one by one along a longitudinal direction of the cross-sectional image with an r-axis as a horizontal axis and a 8-axis as a vertical axis. The control unit 41 may simply obtain the centroid of the extracted point cloud on the inside surface, but in that case, since the point cloud is not uniformly sampled over the inside surface, a centroid position shifts. Therefore, in the present embodiment, the control unit 41 calculates the convex hull of the extracted point cloud on the inside surface, and calculates a centroid position Cₙ=(Cₓ, C_{y}) by using a formula for obtaining the centroid of a polygon as follows. However, in the following formula (Mathematical formula 1), it is assumed that n vertices (x₀, y₀), (x₁, y₁), . . . , (xₙ₋₁, yₙ₋₁) are present on the convex hull counterclockwise as the point cloud on the inside surface as illustrated in FIG. 11, and (xₙ, yₙ) is regarded as (x₀, y₀).

$${C}_{x} = \frac{1}{6 A} {\sum }_{i = 0}^{n - 1} ( {x}_{i} + {x}_{i + 1} ) ( {x}_{i} {y}_{i + 1} - {x}_{i + 1} {y}_{i} ) \\ {C}_{y} = \frac{1}{6 A} {\sum }_{i = 0}^{n - 1} ( {y}_{i} + {y}_{i + 1} ) ( {x}_{i} {y}_{i + 1} - {x}_{i + 1} {y}_{i} ) \\ A = \frac{1}{2} {\sum }_{i = 0}^{n - 1} ( {x}_{i} {y}_{i + 1} - {x}_{i + 1} {y}_{i} ) \qquad Mathematical formula 1$$

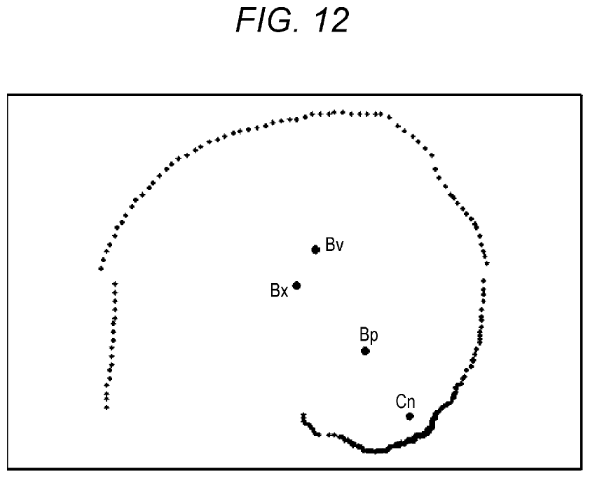

The centroid positions obtained as results are illustrated in FIG. 12. In FIG. 12, a point Cn is the center of the cross-sectional image. A point Bp is a centroid of the point cloud on the inside surface. A point By is a centroid of the vertices of the polygon. A point Bx is a centroid of the polygon serving as a convex hull.

As a method of calculating the centroid position of the blood vessel, a method other than the method of calculating the centroid position of the polygon serving as the convex hull may be used. For example, with respect to an original cross-sectional image that is not binarized, a method of calculating a center position of the maximum circle that falls within the main blood vessel as the centroid position may be used. Alternatively, with respect to the binarized cross-sectional image having the r-axis as the horizontal axis and the 8-axis as the vertical axis, a method of calculating an average position of pixels in a main blood vessel region as the centroid position may be used. The same method as described above may also be used when the biological tissue 60 is not a blood vessel.

In S114, the control unit 41 of the image processing device 11 smooths calculation results of the centroid positions in S113.

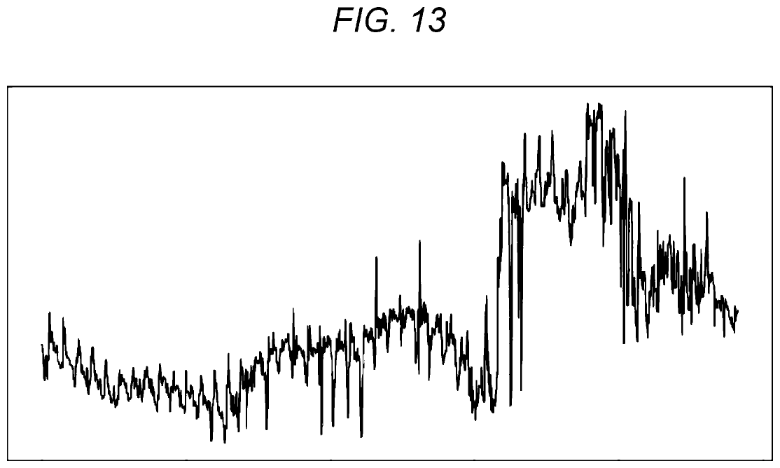

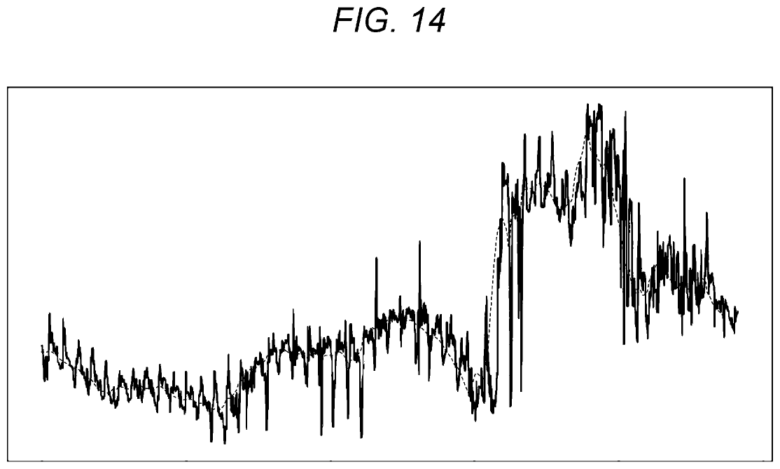

As illustrated in FIG. 13, when the calculation results of the centroid positions are viewed as a time function, it can be seen that an influence of pulsation is relatively large. Therefore, in the present embodiment, the control unit 41 of the image processing device 11 smooths the calculation results of the centroid positions by using moving averages as indicated by a broken line in FIG. 14.

As a smoothing method, a method other than the movement average may be used. For example, exponential smoothing method, kernel method, local regression, Ramer-Douglas-Peucker algorithm, Savitzky-Golay method, smoothed spline, or stretched grid method (SGM) may be used. Alternatively, a method of executing the fast Fourier transform and then removing a high-frequency component may be used. Alternatively, Kalman filter or a low-pass filter such as Butterworth filter, Chebyshev filter, digital filter, elliptical filter, or Kolmogorov-Zurbenko (KZ) filter may be used.

Simple smoothing may cause the centroid positions to enter the tissue. In this case, the control unit 41 may divide the calculation results of the centroid position, in the longitudinal direction of the lumen 63 of the biological tissue 60, according to positions of the plurality of lateral cross sections of the lumen 63 of the biological tissue 60, and may smooth each of the divided calculation results. That is, when a curve of the centroid positions as indicated by the broken line in FIG. 14 overlaps a tissue region, the control unit 41 may divide the curve of the centroid positions into a plurality of sections and execute individual smoothing for each section. Alternatively, the control unit 41 may adjust a degree of smoothing to be executed on the calculation results of the centroid positions according to the positions of the plurality of lateral cross sections of the lumen 63 of the biological tissue 60 in the longitudinal direction of the lumen 63 of the biological tissue 60. That is, when the curve of the centroid positions as indicated by the broken line in FIG. 14 overlaps the tissue region, the control unit 41 may decrease the degree of smoothing to be executed for a part of the sections including the overlapping points.

In S115, as illustrated in FIG. 4, the control unit 41 of the image processing device 11 sets two planes intersecting at the single line Lb passing through the centroid positions calculated in S113, as cutting planes P1 and P2. In the present embodiment, the control unit 41 smooths the calculation results of the centroid positions in S114, and then sets the cutting planes P1 and P2, but the processing of S114 may be omitted.

Specifically, the control unit 41 of the image processing device 11 sets a curve of the centroid positions obtained as a result of the smoothing in S114 as the line Lb. The control unit 41 sets two planes intersecting at the set line Lb and including the two respective straight lines L1 and L2 set in S112 as the cutting planes P1 and P2. The control unit 41 identifies three-dimensional coordinates intersecting with the cutting planes P1 and P2 of the biological tissue 60 in the latest three-dimensional data 52 stored in the storage unit 42 as the three-dimensional coordinates of an edge of the opening exposing the lumen 63 of the biological tissue 60 in the three-dimensional image 53. The control unit 41 stores the identified three-dimensional coordinates in the storage unit 42.

In S116, the control unit 41 of the image processing device 11 forms, in the three-dimensional data 52, a region that is interposed between the cutting planes P1 and P2 in the three-dimensional image 53 and that exposes the lumen 63 of the biological tissue 60, as a cutting region 62.

Specifically, the control unit 41 of the image processing device 11 sets a portion identified by the three-dimensional coordinates stored in the storage unit 42 in the latest three-dimensional data 52 stored in the storage unit 42 to be hidden or transparent when the three-dimensional image 53 is displayed on the display 16. That is, the control unit 41 forms the cutting region 62 in accordance with the region 65 set in S112.

In S117, the control unit 41 of the image processing device 11 displays the three-dimensional data 52 in which the cutting region 62 is formed in S116 on the display 16, as the three-dimensional image 53. The control unit 41 displays the two-dimensional image 56 representing the cross section 64 indicated by the tomographic data 51 newly acquired by the sensor and represented by the cross-sectional image displayed on the display 16 in S103 and the region 65 corresponding to the cutting region 62 in the cross section 64 on the display 16 together with the three-dimensional image 53.

Specifically, the control unit 41 of the image processing device 11 generates the two-dimensional image 56 as illustrated in FIG. 2 by processing the latest cross-sectional image among the cross-sectional images of the biological tissue 60 included in the tomographic data 51 stored in the storage unit 42. The control unit 41 generates the three-dimensional image 53 as illustrated in FIG. 2 in which a portion identified by the three-dimensional coordinates stored in the storage unit 42 is hidden or transparent. The control unit 41 displays the two-dimensional image 56 and the three-dimensional image 53 that are generated, on the display 16 via the output unit 45.

In the present embodiment, as illustrated in FIG. 2, the control unit 41 of the image processing device 11 generates, as the two-dimensional image 56, an image representing the color of the region 65 corresponding to the cutting region 62 in a color different from that of the remaining region. For example, it is conceivable to change a white portion in a general IVUS image to red in the region 65.

In S118, if there is an operation of setting the cutting region 62 as a change operation by the user, the processing of S119 is executed. If there is no change operation by the user, processing of S120 is executed.

In S119, the control unit 41 of the image processing device 11 receives, via the input unit 44, the operation of setting the cutting region 62, similarly to the processing in S112. Then, the processing in and after S115 is executed.

If the tomographic data 51 is updated in S120, the processing in S121 and S122 is executed. If the tomographic data 51 is not updated, the presence or absence of the change operation by the user is confirmed again in S118.

In S121, similarly to the processing in S101 or S107, the control unit 41 of the image processing device 11 processes the signal input from the probe 20 to newly generate cross-sectional images of the biological tissue 60, thereby acquiring the tomographic data 51 including at least one new cross-sectional image.

In S122, the control unit 41 of the image processing device 11 updates the three-dimensional data 52 of the biological tissue 60 based on the tomographic data 51 acquired in S121. Thereafter, the processing in and after S113 is executed. In S122, it is preferable to update only data at a location corresponding to the updated tomographic data 51. Accordingly, the data processing amount when generating the three-dimensional data 52 can be reduced, and the real-time property of data processing in and after S113.

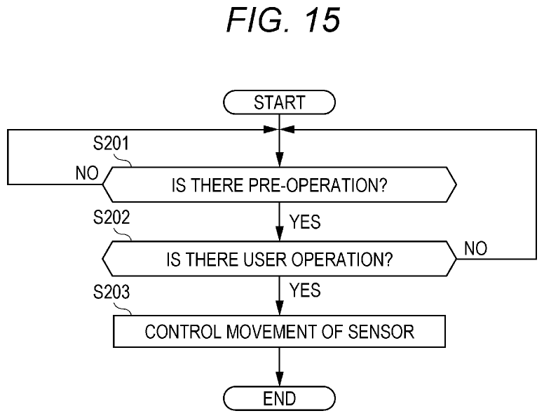

The operation of the image processing system 10 according to the present embodiment will be further described with reference to FIG. 15.

In S201, when a pre-operation of clicking or tapping the first graphic element 87 while pressing a predetermined key is performed, the control unit 41 of the image processing device 11 receives the pre-operation via the input unit 44. The pre-operation can be, for example, an operation of clicking the first graphic element 87 using the mouse 15 while pressing the shift key of the keyboard 14, or an operation of tapping the first graphic element 87 using the touch screen disposed integrally with the display 16 while pressing the shift key of the keyboard 14. An erroneous operation can be prevented by including an operation of pressing a specific key such as a shift key in the pre-operation.

In S202, when an operation of dragging the first graphic element 87 and dropping the first graphic element 87 at the position corresponding to the movement destination of the sensor is performed as a user operation, the control unit 41 of the image processing device 11 receives the user operation via the input unit 44. The user operation can be, for example, an operation of dragging and dropping the first graphic element 87 using the mouse 15 or the touch screen disposed integrally with the display 16.

In the present embodiment, in a case where a pre-operation is performed before a user operation, the control unit 41 of the image processing device 11 receives the user operation. The control unit 41 does not receive a user operation until a pre-operation is performed. Even if a pre-operation is performed, if a user operation is not performed within a certain period of time, the control unit 41 does not receive the user operation until the pre-operation is performed again. Therefore, an erroneous operation can be reliably prevented.

In S203, the control unit 41 of the image processing device 11 controls the movement of the sensor via the communication unit 43 according to the user operation, and changes the relative position of the first graphic element 87 via the output unit 45 to cause the first graphic element 87 to represent the position of the sensor after the movement.

Specifically, the control unit 41 of the image processing device 11 performs control so that the first graphic element 87a representing the position of the sensor after the movement is displayed at the position where the first graphic element 87 is dropped on the screen 80. At the same time, the control unit 41 calculates the distance from the position of the first graphic element 87 before dragging to the position of the first graphic element 87 after dropping, that is, the position of the first graphic element 87a. The control unit 41 calculates the movement distance of the sensor corresponding to the calculated distance using a predefined conversion formula or conversion. The control unit 41 transmits a signal instructing the scanner unit 31 to move forward or backward by the calculated movement distance to the drive unit 13 via the image diagnostic interface. In a case where the first graphic element 87 has been dragged upward in the user operation, a signal is sent instructing the scanner unit 31 to move forward. In the drive unit 13, the slide motor 38 rotates forward according to the transmitted signal, and the scanner unit 31 moves forward. That is, the same operation as when the forward switch included in the switch group 39 is pressed is performed. In a case where the first graphic element 87 has been dragged downward in the user operation, a signal is sent instructing the scanner unit 31 to be move backward. In the drive unit 13, the slide motor 38 reversely rotates according to the transmitted signal, and the scanner unit 31 moves backward. That is, the same operation as when the pull-back switch included in the switch group 39 is pressed is performed.

In the present embodiment, the control unit 41 of the image processing device 11 controls the movement of the sensor after the first graphic element 87 is dropped. Even if the first graphic element 87 is dragged, the control unit 41 does not start movement of the sensor until the first graphic element 87 is dropped.

The control unit 41 of the image processing device 11 may adjust the movement speed of the sensor and causes the sensor to reach the movement destination in a certain period of time regardless of the distance from the position of the first graphic element 87 before dragging to the position of the first graphic element 87 after dropping. For example, the control unit 41 may adjust the forward/backward movement speed of the scanner unit 31 such that the forward/backward movement of the scanner unit 31 ends, for example, in about 2 seconds regardless of the distance by which the first graphic element 87 is dragged.

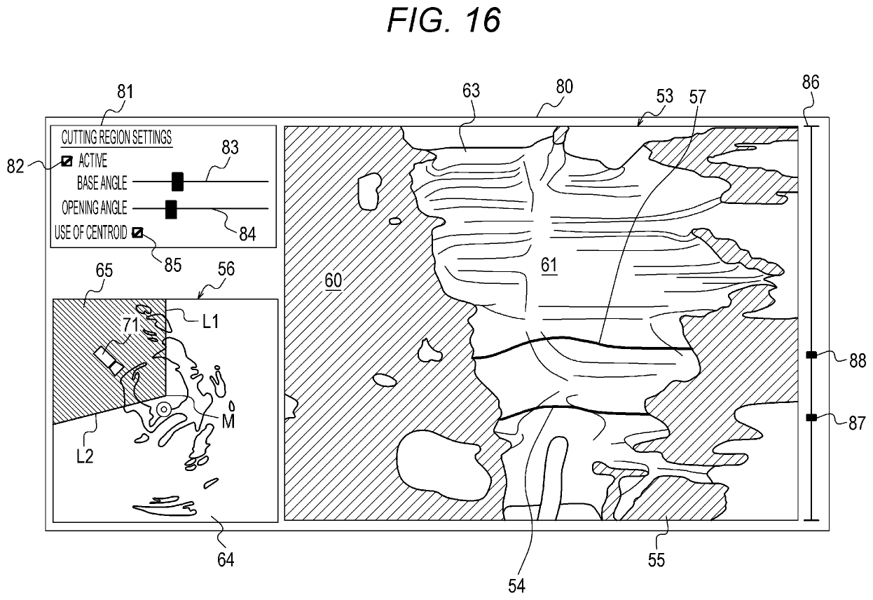

In the present embodiment, as illustrated in FIG. 16, the control unit 41 of the image processing device 11 colors, using a predetermined color, at least voxels representing the inside surface 61 of the biological tissue 60 or voxels adjacent to the voxels representing the inside surface 61 and representing the lumen 63 in a third voxel group 57 at a position corresponding to the position after displacement of the first graphic element 87 in the three-dimensional image 53 every time the first graphic element 87 is displaced by the first graphic element 87 being dragged. For example, assuming that a drag operation is currently being performed and a drop operation is not being performed, as illustrated in FIG. 16, voxels representing the inside surface 61 of the biological tissue 60 in the first voxel group 54 corresponding to the current position of the sensor are colored using a first color such as green. In the third voxel group 57 at the position corresponding to the displaced position of the first graphic element 87, that is, the position of a first graphic element 88 being dragged, voxels representing the inside surface 61 of the biological tissue 60 are colored using a second color, for example, such as orange. That is, the line of the second color is displayed together with the line of the first color. The first graphic element 88 represents the position of the sensor after temporary movement by being located at the same height as the third voxel group 57. The first graphic element 87 representing the current position of the sensor is displayed in a different color than the first graphic element 88 being dragged. For example, the first graphic element 87 representing the current position of the sensor is displayed in the first color, and the first graphic element 88 being dragged is displayed in the second color. In a modification of the present embodiment, the line of the first color may be hidden. The first graphic element 87 representing the current position of the sensor may also be hidden.

The control unit 41 of the image processing device 11 may further display, on the display 16, a two-dimensional image representing a cross section indicated by the tomographic data 51 acquired by the sensor at the position corresponding to the position of the first graphic element 87 after the displacement every time the first graphic element 87 is displaced by the first graphic element 87 being dragged. For example, a recent cross-sectional image corresponding to the position of the first graphic element 88 being dragged may be displayed in a balloon at that position.

While the first graphic element 87 is being dragged, the control unit 41 of the image processing device 11 may further display, on the display 16, a numerical value indicating the movement distance of the sensor corresponding to the distance from the position of the first graphic element 87 before being dragged to the position of the first graphic element 87 after being dragged, that is, the position of the first graphic element 88 being dragged. For example, it is assumed that it is known that the sensor is located in the vicinity of one end of the oval fossa in the longitudinal direction and that the longitudinal length of the oval fossa is, for example, about 10 mm. In this case, the sensor can be relatively easily and reliably move to the vicinity of the other end of the oval fossa in the longitudinal direction by dragging the first graphic element 87 such that the numerical value displayed on the screen 80 is 10 mm and then dropping the first graphic element 87. In the case of cauterizing a location at a certain distance, such as 10 mm from the root of the superior vena cava (SVC) by ablation, the sensor can be easily and reliably moved to a desired position.

In a modification of the present embodiment, the control unit 41 of the image processing device 11 may control the movement of the sensor while the first graphic element 87 is being dragged, instead of controlling the movement of the sensor after the first graphic element 87 is dropped. The control unit 41 may start the movement of the sensor at the time when the first graphic element 87 starts to be dragged. For example, the control unit 41 may adjust the forward/backward movement speed of the scanner unit 31 according to the speed at which the first graphic element 87 is dragged. That is, the control unit 41 may increase the forward/backward movement speed of the scanner unit 31 as the speed at which the first graphic element 87 is dragged increases.

In this modification, the control unit 41 of the image processing device 11 may limit the speed at which the first graphic element 87 is dragged according to the upper limit of the movement speed of the sensor. For example, the control unit 41 may perform adjustment such that the forward/backward movement speed of the scanner unit 31 does not exceed, for example, 40 mm/second no matter how fast the speed at which the first graphic element 87 is dragged.

As described above, in the present embodiment, the control unit 41 of the image processing device 11 displays an image representing the biological tissue 60 on the display 16 based on the tomographic data 51 acquired by the sensor moving in the lumen 63 of the biological tissue 60, and displays an element representing the position of the sensor on the screen 80 same as the image on the display 16. When a user operation of specifying a movement destination of the sensor is performed on the screen 80, the control unit 41 controls movement of the sensor according to the user operation, and changes the relative position of the element to cause the position of the sensor after the movement to be represented.

According to the present embodiment, the sensor can be operated on the screen 80. Therefore, for example, in a case where a procedure such as ablation using IVUS is performed, a doctor performing the procedure while operating a catheter can relatively easily perform a pull-back operation on the screen 80. Alternatively, a clinical technician operating an IVUS system while observing the display 16 can relatively easily perform a pull-back operation on the screen 80. Therefore, a procedure can be relatively easily performed even in a state where the number of staff members is relatively small.

In a modification of the present embodiment, the user operation may be a click or tap operation instead of a drag and drop operation. That is, the processing in S201 may be omitted, and in S202, the control unit 41 of the image processing device 11 may receive, as a user operation, an operation of clicking or tapping a position away from the first graphic element 87 by a distance corresponding to the movement distance of the sensor via the input unit 44. In this case, the user operation can be, for example, an operation of clicking any position of the second graphic element 86 using the mouse 15 or an operation of tapping any position of the second graphic element 86 using the touch screen disposed integrally with the display 16.

In this modification, the control unit 41 of the image processing device 11 may adjust the movement speed of the sensor and causes the sensor to reach the movement destination in a certain period of time regardless of the distance from the position of the first graphic element 87 before clicking or tapping to the position of the clicking or the tapping. For example, the control unit 41 may adjust the forward/backward movement speed of the scanner unit 31 such that the forward/backward movement of the scanner unit 31 ends, for example, in about 2 seconds regardless of the clicked or tapped position.

In a modification of the present embodiment, the control unit 41 of the image processing device 11 may receive an operation of setting a start point and an end point via the input unit 44. Then, the control unit 41 may control the movement of the sensor such that the sensor repeatedly reciprocates between the set start point and end point.

In S101, instead of manually performing the pull-back operation, the operation illustrated in FIG. 15 may be applied to perform the pull-back operation on the screen 80.

In a modification of the present embodiment, the user operation may be an operation of specifying the movement destination of the sensor by inputting a numerical value of the movement distance of the sensor on the screen 80. The user operation may include an operation of selecting a movement direction of the sensor.

In the present embodiment, the control unit 41 of the image processing device 11 displays, as the three-dimensional image 53, the three-dimensional data 52 representing the biological tissue 60 on the display 16. The control unit 41 forms, in the three-dimensional data 52, the cutting region 62 for exposing the lumen 63 of the biological tissue 60 in the three-dimensional image 53. The control unit 41 displays the two-dimensional image 56 representing the cross section 64 of the biological tissue 60 and the region 65 corresponding to the cutting region 62 in the cross section 64 on the display 16 together with the three-dimensional image 53.

According to the present embodiment, how a part of the structure of the biological tissue 60 is cut can be indicated. Therefore, the user can grasp from the two-dimensional image 56 what type of structure the portion of the biological tissue that is cut and not displayed in the three-dimensional image 53 is. For example, in a case where the user is an operator, an operation on the inside of the biological tissue 60 can be relatively easily performed.

In the present embodiment, the control unit 41 of the image processing device 11 generates and updates the three-dimensional data 52 representing the biological tissue 60 based on the tomographic data 51 acquired by the sensor that acquires the tomographic data 51 of the biological tissue 60 while moving in the lumen 63 of the biological tissue 60. The control unit 41 displays the three-dimensional data 52 as the three-dimensional image 53 on the display 16. In the three-dimensional image 53, the control unit 41 colors at least voxels representing the inside surface 61 of the biological tissue 60 or voxels adjacent to the voxels representing the inside surface 61 and representing the lumen 63 in the first voxel group 54 corresponding to the cross section 64 indicated by the tomographic data 51 newly acquired by the sensor separately from the second voxel group 55 corresponding to other cross sections of the biological tissue 60.

According to the present embodiment, which portion in the three-dimensional image 53 the cross section 64 of the biological tissue 60 indicated by the tomographic data 51 newly acquired by the sensor corresponds to can be indicated. Therefore, it is easy for the user observing the lumen 63 of the biological tissue 60 using the three-dimensional image 53 to grasp which portion in the three-dimensional image 53 information currently obtained by the sensor, that is, the latest information corresponds to.

The present disclosure is not limited to the above-described embodiment. For example, two or more blocks described in the block diagrams may be integrated, or one block may be divided. Instead of executing a plurality of steps or processes described in the flowchart in time series according to the description, the steps or processes may be executed in parallel or in a different order according to the processing capability of the device that executes each step or process or as necessary. In addition, modifications can be made within a scope not departing from the gist of the present disclosure.

For example, the processing of S201 may be omitted. That is, in S202, the control unit 41 of the image processing device 11 may receive a user operation regardless of whether a pre-operation is performed before the user operation.

The detailed description above describes embodiments of an image processing device, an image processing system, an image display method, and an image processing program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An image processing device configured to display an image representing a biological tissue on a display and to display an element representing a position of a sensor on a screen same as the image on the display, based on tomographic data acquired by the sensor moving in a lumen of the biological tissue, the image processing device comprising
a control unit configured to, when a user operation of specifying a movement destination of the sensor is performed on the screen, control movement of the sensor according to the user operation, and change a relative position of the element to cause a position of the sensor after movement to be represented.

2. The image processing device according to claim 1, wherein the control unit is configured to receive the user operation in a case in which a pre-operation of clicking or tapping the element while pressing a predetermined key is performed before the user operation.

3. The image processing device according to claim 1, wherein the control unit is configured to receive, as the user operation, an operation of dragging the element and dropping the element at a position corresponding to a movement destination of the sensor.

4. The image processing device according to claim 3, wherein the control unit is configured to control movement of the sensor after the element is dropped.

5. The image processing device according to claim 4, wherein the control unit is configured to adjust a movement speed of the sensor, and cause the sensor to reach a movement destination in a certain period of time regardless of a distance from a position of the element before dragging to a position of the element after dropping.

6. The image processing device according to claim 3, wherein the control unit is configured to control movement of the sensor while the element is being dragged.

7. The image processing device according to claim 6, wherein the control unit is configured to limit a speed at which the element is dragged according to an upper limit of a movement speed of the sensor.

8. The image processing device according to claim 3, wherein the control unit is configured to further display, on the display, a two-dimensional image representing a cross section indicated by tomographic data acquired by the sensor at a position corresponding to a position of the element after displacement every time the element is displaced by the element being dragged.

9. The image processing device according to claim 3, wherein the control unit is configured to color, using a predetermined color, at least a voxel representing an inside surface of the biological tissue or a voxel adjacent to a voxel representing a corresponding inside surface and representing the lumen in a voxel group at a position corresponding to a position after displacement of the element in a three-dimensional image that is the image every time the element is displaced by the element being dragged.

10. The image processing device according to claim 3, wherein the control unit is configured to further display, on the display, a numerical value indicating a movement distance of the sensor corresponding to a distance from a position of the element before dragging to a position of the element after dragging while the element is being dragged.

11. The image processing device according to claim 1, wherein the control unit is configured to receive, as the user operation, an operation of clicking or tapping a position away from the element by a distance corresponding to a movement distance of the sensor.

12. The image processing device according to claim 11, wherein the control unit is configured to adjust a movement speed of the sensor, and cause the sensor to reach a movement destination in a certain period of time regardless of a distance from a position of the element before clicking or tapping to a position of clicking or tapping.

13. The image processing device according to claim 1, wherein the control unit is configured to combine a first graphic element that is the element and a second graphic element representing a movement range of the sensor and display the first graphic element and the second graphic element on the display.

14. The image processing device according to claim 13, wherein the control unit is configured to display the second graphic element on the display in a direction in which a longitudinal direction of the lumen in a three-dimensional image that is the image is parallel to a longitudinal direction of the second graphic element.

15. The image processing device according to claim 1, wherein, in a three-dimensional image that is the image, the control unit is configured to color, as the element, at least a voxel representing an inside surface of the biological tissue or a voxel adjacent to a voxel representing a corresponding inside surface and representing the lumen in a first voxel group corresponding to a cross section indicated by tomographic data newly acquired by the sensor separately from a second voxel group corresponding to another cross section of the biological tissue.

16. An image processing system comprising:
the image processing device according to claim 1; and
a probe including the sensor.

17. The image processing system according to claim 16, further comprising:
the display.

18. An image display method of displaying an image representing a biological tissue on a display and displaying an element representing a position of a sensor on a screen same as the image on the display, based on tomographic data acquired by the sensor moving in a lumen of the biological tissue, the image display method comprising:
when a user operation of specifying a movement destination of the sensor is performed on the screen, controlling, by a computer, movement of the sensor according to the user operation; and
changing, by the computer, a relative position of the element to cause a position of the sensor after movement to be represented.

19. The image display method according to claim 18, further comprising:
receiving, by the computer, the user operation in a case in which a pre-operation of clicking or tapping the element while pressing a predetermined key is performed before the user operation; or
receiving, by the computer, as the user operation, an operation of dragging the element and dropping the element at a position corresponding to a movement destination of the sensor.

20. A non-transitory computer-readable medium storing an image processing program that causes a computer that displays an image representing a biological tissue on a display and displays an element representing a position of a sensor on a screen same as the image on the display, based on tomographic data acquired by the sensor moving in a lumen of the biological tissue to execute a process:
when a user operation of specifying a movement destination of the sensor is performed on the screen, controlling movement of the sensor according to the user operation; and
changing a relative position of the element to cause a position of the sensor after movement to be represented.
